SEQUENCE LISTING (66 sequences)

SEQ ID NO: 1 - RNA, 22 nt, Artificial Sequence
  synthetic miR-200a miRNA
uaacacugucugguaacgaugu

SEQ ID NO: 2 - RNA, 23 nt, Artificial Sequence
  synthetic hsa-miR-125a miRNA, miR-125a miRNA
ucccugagacccuuuaaccugug

SEQ ID NO: 3 - RNA, 22 nt, Artificial Sequence
  synthetic hsa-miR-16 miRNA
uagcagcacguaaauauuggcg

SEQ ID NO: 4 - RNA, 22 nt, Artificial Sequence
  synthetic Let-7b miRNA
ugagguaguagguugugugguu

SEQ ID NO: 5 - RNA, 23 nt, Artificial Sequence
  synthetic hsa-miR-19b miRNA
ugugcaaauccaugcaaaacuga

SEQ ID NO: 6 - RNA, 21 nt, Artificial Sequence
  synthetic hsa-miR-26a miRNA
uucaaguaauccaggauaggc

SEQ ID NO: 7 - RNA, 22 nt, Artificial Sequence
  synthetic hsa-miR-24 miRNA
uggcucaguucagcaggaacag

SEQ ID NO: 8 - RNA, 23 nt, Artificial Sequence
  synthetic hsa-miR-30c miRNA
uguaaacauccuacacucucagc

SEQ ID NO: 9 - RNA, 22 nt, Artificial Sequence
  synthetic hsa-miR-26b miRNA
uucaaguaauucaggauagguu

SEQ ID NO: 10 - RNA, 22 nt, Artificial Sequence
  synthetic hsa-miR-30a-3p miRNA
cuuucagucggauguuugcagc

SEQ ID NO: 11 - RNA, 22 nt, Artificial Sequence
  synthetic hsa-miR-30e-3p miRNA
cuuucagucggauguuuacagc

SEQ ID NO: 12 - RNA, 20 nt, Artificial Sequence
  synthetic hsa-miR-30e-5p miRNA
uguaaacauccuugacugga

SEQ ID NO: 13 - RNA, 21 nt, Artificial Sequence
  synthetic hsa-miR-92 miRNA
uauugcacuugucccggccug

SEQ ID NO: 14 - RNA, 22 nt, Artificial Sequence
  synthetic hsa-miR-146a miRNA
ugagaacugaauuccauggguu

SEQ ID NO: 15 - RNA, 21 nt, Artificial Sequence
  synthetic hsa-miR-140 miRNA
agugguuuuacccuaugguag

SEQ ID NO: 16 - RNA, 22 nt, Artificial Sequence
  synthetic hsa-miR-146b miRNA
ugagaacugaauuccauaggcu

SEQ ID NO: 17 - RNA, 22 nt, Artificial Sequence
  hsa-miR-155 miRNA
uuaaugcuaaucgugauagggg

SEQ ID NO: 18 - RNA, 22 nt, Artificial Sequence
  synthetic hsa-miR-150 miRNA
ucucccaacccuuguaccagug

SEQ ID NO: 19 - RNA, 23 nt, Artificial Sequence
  hsa-miR-181a miRNA
aacauucaacgcugucggugagu

SEQ ID NO: 20 - RNA, 22 nt, Artificial Sequence
  synthetic hsa-miR-181b miRNA
aacauucauugcugucgguggg

SEQ ID NO: 21 - RNA, 22 nt, Artificial Sequence
  synthetic hsa-miR-181c miRNA
aacauucaaccugucggugagu

SEQ ID NO: 22 - RNA, 24 nt, Artificial Sequence
  synthetic hsa-miR-181d miRNA
aacauucauuguugucgguggguu

SEQ ID NO: 23 - RNA, 22 nt, Artificial Sequence
  synthetic hsa-miR-191 miRNA
caacggaaucccaaaagcagcu

SEQ ID NO: 24 - RNA, 21 nt, Artificial Sequence
  synthetic hsa-miR-195 miRNA
uagcagcacagaaauauuggc

SEQ ID NO: 25 - RNA, 22 nt, Artificial Sequence
  synthetic hsa-miR-200c miRNA
uaauacugccggguaaugaugg

SEQ ID NO: 26 - RNA, 22 nt, Artificial Sequence
  synthetic hsa-miR-197 miRNA
uucaccaccuucuccacccagc

SEQ ID NO: 27 - RNA, 22 nt, Artificial Sequence
  synthetic hsa-miR-203 miRNA
gugaaauguuuaggaccacuag

SEQ ID NO: 28 - RNA, 24 nt, Artificial Sequence
  synthetic hsa-miR-222 miRNA
agcuacaucuggcuacugggucuc

SEQ ID NO: 29 - RNA, 21 nt, Artificial Sequence
  synthetic hsa-miR-223 miRNA
ugucaguuugucaaauacccc

SEQ ID NO: 30 - RNA, 23 nt, Artificial Sequence
  synthetic hsa-miR-320 miRNA
aaaagcuggguugagagggcgaa

SEQ ID NO: 31 - RNA, 24 nt, Artificial Sequence
  synthetic hsa-miR-342 miRNA
ucucacacagaaaucgcacccguc

SEQ ID NO: 32 - RNA, 22 nt, Artificial Sequence
  synthetic hsa-miR-375 miRNA
uuuguucguucggcucgcguga

SEQ ID NO: 33 - RNA, 22 nt, Artificial Sequence
  synthetic hsa-miR-184 miRNA
uggacggagaacugauaagggu

SEQ ID NO: 34 - RNA, 22 nt, Artificial Sequence
  synthetic hsa-miR-34c miRNA
aggcaguguaguuagcugauug

SEQ ID NO: 35 - RNA, 22 nt, Artificial Sequence
  synthetic hsa-miR-137 miRNA
uauugcuuaagaauacgcguag

SEQ ID NO: 36 - RNA, 23 nt, Artificial Sequence
  synthetic hsa-miR-372 miRNA
aaagugcugcgacauuugagcgu

SEQ ID NO: 37 - RNA, 22 nt, Artificial Sequence
  synthetic hsa-miR-124a miRNA
uuaaggcacgcggugaaugcca

SEQ ID NO: 38 - RNA, 22 nt, Artificial Sequence
  synthetic hsa-miR-21 miRNA
uagcuuaucagacugauguuga

SEQ ID NO: 39 - RNA, 20 nt, Artificial Sequence
  synthetic hsa-miR-124b miRNA
uuaaggcacgcggugaaugc

SEQ ID NO: 40 - RNA, 21 nt, Artificial Sequence
  synthetic hsa-miR-31 miRNA
ggcaagaugcuggcauagcug

SEQ ID NO: 41 - RNA, 22 nt, Artificial Sequence
  synthetic hsa-miR-128a miRNA
ucacagugaaccggucucuuuu

SEQ ID NO: 42 - RNA, 22 nt, Artificial Sequence
  synthetic hsa-miR-34b miRNA
aggcagugucauuagcugauug

SEQ ID NO: 43 - RNA, 22 nt, Artificial Sequence
  synthetic hsa-miR-154 miRNA
aaucauacacgguugaccuauu

SEQ ID NO: 44 - RNA, 22 nt, Artificial Sequence
  synthetic hsa-miR-197 miRNA
uucaccaccuucuccacccagc

SEQ ID NO: 45 - RNA, 22 nt, Artificial Sequence
  synthetic hsa-miR-132 miRNA
uaacagucuacagccauggucg

SEQ ID NO: 46 - RNA, 20 nt, Artificial Sequence
  synthetic hsa-miR-147 miRNA
guguguggaaaugcuucugc

SEQ ID NO: 47 - RNA, 21 nt, Artificial Sequence
  synthetic hsa-miR-325 miRNA
ccuaguagguguccaguaagu

SEQ ID NO: 48 - RNA, 22 nt, Artificial Sequence
  synthetic hsa-miR-181c miRNA
aacauucaaccugucggugagu

SEQ ID NO: 49 - RNA, 19 nt, Artificial Sequence
  synthetic hsa-miR-198 miRNA
gguccagaggggagauagg

SEQ ID NO: 50 - RNA, 22 nt, Artificial Sequence
  synthetic hsa-miR-338 miRNA
acauagaggaaauuccacguuu

SEQ ID NO: 51 - RNA, 24 nt, Artificial Sequence
  synthetic hsa-miR-17-5p miRNA
caaagugcuuacagugcagguagu

SEQ ID NO: 52 - RNA, 22 nt, Artificial Sequence
  synthetic hsa-miR-104 miRNA
ucaacaucagucugauaagcua

SEQ ID NO: 53 - RNA, 21 nt, Artificial Sequence
  synthetic hsa-miR-134 miRNA
ugugacugguugaccagaggg

SEQ ID NO: 54 - RNA, 22 nt, Artificial Sequence
  synthetic hsa-miR-213 miRNA
accaucgaccguugauuguacc

SEQ ID NO: 55 - RNA, 22 nt, Artificial Sequence
  synthetic hsa-miR-133a miRNA
uugguccccuucaaccagcugu

SEQ ID NO: 56 - RNA, 22 nt, Artificial Sequence
  synthetic hsa-miR-99a miRNA
aacccguagauccgaucuugug

SEQ ID NO: 57 - RNA, 22 nt, Artificial Sequence
  synthetic hsa-miR-194 miRNA
uguaacagcaacuccaugugga

SEQ ID NO: 58 - RNA, 21 nt, Artificial Sequence
  synthetic hsa-miR-133b miRNA
uugguccccuucaaccagcua

SEQ ID NO: 59 - RNA, 21 nt, Artificial Sequence
  synthetic hsa-miR-219miRNA
ugauuguccaaacgcaauucu

SEQ ID NO: 60 - RNA, 22 nt, Artificial Sequence
  synthetic hsa-miR-100 miRNA
aacccguagauccgaacuugug

SEQ ID NO: 61 - RNA, 22 nt, Artificial Sequence
  synthetic hsa-miR-125b miRNA
ucccugagacccuaacuuguga

SEQ ID NO: 62 - RNA, 21 nt, Artificial Sequence
  synthetic hsa-miR-26b miRNA
uucaaguaauucaggauaggu

SEQ ID NO: 63 - RNA, 17 nt, Artificial Sequence
  synthetic hsa-miR-138 miRNA
agcugguguugugaauc

SEQ ID NO: 64 - RNA, 22 nt, Artificial Sequence
  synthetic hsa-miR-149 miRNA
ucuggcuccgugucuucacucc

SEQ ID NO: 65 - RNA, 23 nt, Artificial Sequence
  synthetic hsa-miR-107 miRNA
agcagcauuguacagggcuauca

SEQ ID NO: 66 - RNA, 18 nt, Artificial Sequence
  synthetic hsa-miR-139 miRNA
ucuacagugcacgugucu

(19) United States
(12) Patent Application Publication
(10) Pub. No.: US 2009/0317820 A1
(43) Pub. Date: Dec. 24, 2009

(54) MICRO-RNA PROFILE IN HUMAN SALIVA AND ITS USE FOR DETECTION OF ORAL CANCER

(75) Inventors: David T.W. Wong, Beverly Hills, CA (US); Noh Jin Park, Irvine, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: The Regents of the University of California, Oakland, CA (US)

(21) Appl. No.: 12/468,766

(22) Filed: May 19, 2009

Related U.S. Application Data
(60) Provisional application No. 61/128,237, filed on May 19, 2008.

(51) Int. Cl. C12Q 1/68

(52) U.S. Cl. 435/6

(57) ABSTRACT

The present invention provides for the first time the detection of micro-RNA in human saliva and the correlation between such micro-RNA and oral cancers. The present invention therefore provides methods and kits for diagnosing oral cancers by examining pertinent micro-RNA in saliva.

### CROSS-REFERENCES TO RELATED APPLICATIONS

This application claims priority benefit of U.S. Provisional Application Ser. No. 61/128,237 filed on May 19, 2008, the contents of which are incorporated by reference in their entirety for all purposes.

### STATEMENT AS TO RIGHTS TO INVENTIONS MADE UNDER FEDERALLY SPONSORED RESEARCH OR DEVELOPMENT

This invention was made with Government support of Grant No. RO1 DE 015970 awarded by the NIH. The Government has certain rights in this invention.

### REFERENCE TO A “SEQUENCE LISTING,” A TABLE, OR A COMPUTER PROGRAM LISTING APPENDIX SUBMITTED ON A COMPACT DISK NOT APPLICABLE

### BACKGROUND OF THE INVENTION

Oral squamous cell carcinoma (OSCC) constitutes about 90% of oral cancer incidences. In America, OSCC is the 6th most common cancer. About 8,000 people die from this cancer every year (1). Most common metastatic site for OSCC is cervical lymph node, and further metastasis can be found in lungs and bone (2). The average 5-year survival rate for OSCC is about 50%, and surprisingly this number has not changed in last 3 decades (3). Therefore, to increase the patient survival rate, there is an urgent need for an early detection method for oral cancer. This invention meets this and other related needs.

### BRIEF SUMMARY OF THE INVENTION

In one aspect, the present invention provides a method of diagnosing an oral cancer in a subject. The method includes the steps of: (a) detecting in a saliva sample from the subject the level of a micro RNA (miRNA) selected from the Table 2; and (b) determining whether the level is increased or decreased when compared to a standard control, thereby providing a diagnosis for oral cancer. In some embodiments, step (a) comprises an amplification reaction, for example, a polymerase chain reaction (PCR), especially a reverse transcription (RT)-PCR. In some embodiments, saliva sample is whole saliva or saliva supernatant, whereas the miRNA is selected from the left column (“Whole saliva”) or the right column (“Supernatant saliva”) of Table 2. In some examples, the miRNA is miR-200a, miR-125a, miR-142-3p, or miR-93. In other examples, the miRNA is miR-200a or miR-125a and the miRNA level is decreased from the standard control. In other examples, the miRNA is differentially expressed in squamous cell carcinoma of the tongue. In some embodiments, step (a) of the method comprises contacting the saliva sample with a reagent that specifically hybridizes to the miRNA. The reagent may be a nucleic acid, particularly an RT-PCR primer. The reagent may also contain a detectable label or moiety that permits easy detection. An example of the oral cancers that can be detected by the claimed method is oral squamous cell carcinomas (OSCC).

In another aspect, the present invention provides a method for providing prognosis for oral cancer in a subject. The method includes the steps of: (a) detecting in a saliva sample from the subject the level of a micro RNA (miRNA) selected from the Table 2; and (b) determining whether the level is increased or decreased when compared to a standard control, thereby providing a prognosis for oral cancer. In some embodiments, step (a) comprises an amplification reaction, for example, a polymerase chain reaction (PCR), especially a reverse transcription (RT)-PCR. In some embodiments, saliva sample is whole saliva or saliva supernatant, whereas the miRNA is selected from the left column (“Whole saliva”) or the right column (“Supernatant saliva”) of Table 2. In some examples, the miRNA is miR-200a, miR-125a, miR-142-3p, or miR-93. In other examples, the miRNA is miR-200a or miR-125a and the miRNA level is decreased from the standard control. In other examples, the miRNA is differentially expressed in squamous cell carcinoma of the tongue. In some embodiments, step (a) of the method comprises contacting the saliva sample with a reagent that specifically hybridizes to the miRNA. The reagent may be a nucleic acid, particularly an RT-PCR primer. The reagent may also contain a detectable label or moiety that permits easy detection. An example of the oral cancers that can be detected by the claimed method is oral squamous cell carcinomas.

In yet another aspect, this invention also provides a method for monitoring efficacy of a treatment for oral cancer in a subject. The method includes the steps of: (a) detecting in a saliva sample from the subject the level of a micro RNA (miRNA) selected from the Table 2; and (b) determining whether the level is increased or decreased when compared to a standard control, thereby monitoring efficacy of a treatment for oral cancer. In some embodiments, step (a) comprises an amplification reaction, for example, a polymerase chain reaction (PCR), especially a reverse transcription (RT)-PCR. In some embodiments, saliva sample is whole saliva or saliva supernatant, whereas the miRNA is selected from the left column (“Whole saliva”) or the right column (“Supernatant saliva”) of Table 2. In some examples, the miRNA is miR-200a, miR-125a, miR-142-3p, or miR-93. In other examples, the miRNA is miR-200a or miR-125a and the miRNA level is decreased from the standard control. In other examples, the miRNA is differentially expressed in squamous cell carcinoma of the tongue. In some embodiments, step (a) of the method comprises contacting the saliva sample with a reagent that specifically hybridizes to the miRNA. The reagent may be a nucleic acid, particularly an RT-PCR primer. The reagent may also contain a detectable label or moiety that permits easy detection. An example of the oral cancers that can be detected by the claimed method is oral squamous cell carcinomas.

In another aspect still the invention provides target candidate markers (e.g., markers of Table 2 below, including particularly mature micro RNA: miR-200a, miR-125a, miR-142-3p, or miR-93, and also miRNA differentially expressed in squamous cell carcinoma of the tongue) and a method for identifying a salivary micro RNA (miRNA) marker for a human disease state of interest. In some embodiments, the disease state can be systemic or a localized disease state of the head, neck, oropharyngeal cavity, or tongue. The disease of interest can be a cancer, an autoimmune disease, a metabolic disorder, diabetes, or a neurological disorder.

In this aspect, a saliva sample is obtained from a human subject having the disease state of interest and contacting the sample with a RNAse inhibitor; the miRNA is then amplified to provide nucleic acid amplification products of the miRNA and the amplification products are detected. In order to identify the salivary miRNA marker, the relative amounts of miRNA amplification products detected in the sample from the subject having the disease is compared to the miRNA amplification products detected for a control sample which came from a subject not having the disease state; wherein a differential expression indicates the miRNA is a marker for the human disease of interest. The RNAse in the sample can be inhibited by contacting the sample with RNAlater™. The association of the marker with the disease state is confirmed or demonstrated by comparing the relative amount of miRNA amplification products detected in a plurality of samples from a corresponding plurality of subjects having the disease state to miRNA amplification products detected for a plurality of control samples which come from a corresponding plurality of subjects not having the disease state; thereby identifying whether the miRNA in the saliva sample is differentially expressed between the subjects having the disease and the control subjects. In preferred embodiments of the above, the human saliva sample is a cell-free fluid phase portion of saliva. In such embodiments, the saliva can be stimulated or unstimulated saliva.

In a further aspect, the invention provides methods of diagnosing or providing a prognosis by detecting in a saliva sample from a subject the level of a micro RNA (miRNA) identified to be associated with a disease state of interest and determining whether the level is increased or decreased when compared to a standard control, thereby providing a diagnosis for the disease state. In some embodiments, the miRNA is identified as being associated with the disease state by an above method according to the invention.

In a further aspect, the invention provides a method for monitoring efficacy of a treatment for a disease state in a subject, by detecting in a saliva sample from the subject the level of a micro RNA (miRNA) identified as being associated with a disease state and determining whether the level is increased or decreased when compared to a standard control, thereby monitoring efficacy of a treatment for the disease state. In some embodiments, the disease state is an oral cancer. In other embodiments of the above, the miRNA differentially expressed in the disease state is identified according to a method of the invention cancer.

In some embodiments of the above aspects, the detecting step comprises an amplification reaction (e.g., a polymerase chain reaction (PCR) or a reverse transcription (RT)-PCR)) or contacting the saliva sample with a reagent (nucleic acid, primer, probe) that specifically hybridizes to the miRNA. The reagent can carry a detectable label. The methods can be practiced on whole saliva or a saliva supernatant. In further embodiments of any of the above, the miRNA is miR-200a, miR-125a, miR-142-3p, or miR-93. In other embodiments, the miRNA is miR-200a or miR-125a and the miRNA level is decreased from the standard control.

In still another aspect, the invention provides probes or primers for use in detecting miRNA in saliva wherein the probes have a nucleic acid complementary to a mature miRNA of Table 2 or an miRNA differentially expressed in squamous cell carcinoma of the tongue.

### BRIEF DESCRIPTION OF THE DRAWINGS

FIG. 1: Stability of endogenous and exogenous miRNA in saliva. At time 0, to the supernatant phase of saliva, exogenous miR-124a was added to a final concentration of 50 μM. The saliva was incubated at room temperature for up to 30 minutes. At each time point, 400 μL of saliva was removed for RT-preamp-qPCR of miR-124a and miR-191. The amount of RNA quantified at each time points were normalized to time 0. Triplicate aliquots were removed at each time point. Error bars represent standard deviation.

### DETAILED DESCRIPTION OF THE INVENTION

### Introduction

Saliva has been used as a diagnostic medium for OSCC. Saliva analytes such as proteins and DNA have been used to detect OSCC (1, 4). Our lab showed that thousands of mRNAs are present in saliva, and a panel of these mRNAs can be used for oral cancer detection (5-7). These mRNAs appears to enter the saliva in the oral cavity through various sources including 3 major saliva glands, gingival crevice fluid, and desquamated oral epithelial cells (8). Majority of saliva mRNAs appear to be partially degraded at random positions (9), but these partially degraded mRNAs can still be quantitatively analyzed by various techniques such as microarray and RT-PCR.

miRNAs lin-4 and let-7 were initially discovered in C. elegans as key regulators of the animal development (10). However, the mass mining of miRNAs came in early 2000 (11-13), and the mechanism of miRNA production and its mode of action have been well characterized. miRNAs are transcribed by polymerase II or polymerase III as a part of an intron of MRNA or as an independent gene unit (14, 15). Initially transcribed miRNAs can be several hundred to thousands of nt with a distinct stem-loop structure, which gets cleaved into usually less than 100 nt step loop structure by a type III ribonuclease termed Drosha (16). These pre-miRNAs are then exported out to the cytoplasm via exportin 5, and they go through another round of endonucleolytic cleavage by another type III ribonuclease termed, Dicer (17, 18). The final miRNAs are usually about 18-24 nt. The mature form of miRNAs are bound by a protein complex called RNA-induced-silencing-comple (RISC), which is composed of 4 argonaute family proteins Ago1-4 (19). This active miRNA-RISC complex binds to the target mRNA based on the sequences homology and the usual mode of action is the translation blockage and/or mRNA degradation. Because miRNAs can bind to imperfect complementary target mRNA, it is estimated that one miRNA can bind to more than 100 different mRNAs with different binding efficiency. With about 1000 miRNAs expected to be present in human, it is postulated that about 30% of all mRNAs are post-transcriptionally regulated by miRNAs (20, 21).

Recent mining of hundreds of miRNAs from various organisms, and their functional studies revealed that miRNAs serve important functions in cell growth, differentiation, apoptosis, stress response, immune response, and glucose secretion (22-26). Many research groups showed that miRNAs are differentially expressed in various cancer cells and it appears that miRNAs can be better than mRNAs in clustering different types of solid tumors, suggesting that miRNAs can be used to detect cancer (22). In addition, unlike mRNAs where their expression fold changes in cancer cells are relative small compared to normal cells, many of miRNAs show tens to hundreds fold changes in their expression level in cancer cells compared to normal cells (27).

In this work, we performed global profiling of miRNAs in both whole and spun-down supernatant saliva of healthy subjects and OSCC patients. Our results indicate the relevance of several miRNA to OSCC and the feasibility of monitoring for miRNA in saliva samples.

The present invention thus provides a novel method for the diagnosis of oral cancers, especially OSCC, by detecting one or more miRNA provided in Table 2 or other miRNA associated with squamous cell carcinoma of the tongue, in either whole or supernatant saliva samples. The diagnosis is made based on the quantitative change, either an increase or a decrease, from a control level or baseline. Similarly, changes in relevant miRNA levels can also provide information for a prognosis for an oral cancer, or to indicate therapeutic efficacy of the treatment a patient is receiving for his/her oral cancer.

### DEFINITIONS

“Micro RNAs” or “miRNAs” are single-stranded RNA molecules of about 18-24 nucleotides in length that regulate gene expression. miRNAs are encoded by genes that are transcribed from DNA but not translated into protein (non-coding RNA); instead they are processed from primary transcripts known as pri-miRNA to short stem-loop structures called pre-miRNA and finally to functional miRNA. Mature miRNA molecules are partially complementary to one or more messenger RNA (mRNA) molecules, and their main function is to downregulate gene expression. Various miRNA sequences are provided, for example, in GenBank Accession Nos. MI0000737 and MI0000469 for miR-200a (UMCACUGUCUGGUAACGAUGU) and miR-125a (UCCCUGAGACCCUUUAACCUGUG). In other embodiments, the miRNA is an miRNA selected from the following list:

| | |
|---|---|
| hsa-miR-16 | UAGCAGCACGUAAAUAUUGGCG |
| Let-7b | UGAGGUAGUAGGUUGUGUGGUU |
| hsa-miR-19b | UGUGCAAAUCCAUGCAAAACUGA |
| hsa-miR-26a | UUCAAGUAAUCCAGGAUAGGC |
| hsa-miR-24 | UGGCUCAGUUCAGCAGGAACAG |
| hsa-miR-30c | UGUAAACAUCCUACACUCUCAGC |
| hsa-miR-26b | UUCAAGUAAUUCAGGAUAGGUU |
| hsa-miR-30a-3p | CUUUCAGUCGGAUGUUUGCAGC |
| hsa-miR-30e-3p | CUUUCAGUCGGAUGUUUACAGC |
| hsa-miR-30e-5p | UGUAAACAUCCUUGACUGGA |
| hsa-miR-92 | UAUUGCACUUGUCCCGGCCUG |
| hsa-miR-125a | UCCCUGAGACCCUUUAACCUGUG |
| hsa-miR-146a | UGAGAACUGAAUUCCAUGGGUU |
| hsa-miR-140 | AGUGGUUUUACCCUAUGGUAG |
| hsa-miR-146b | UGAGAACUGAAUUCCAUAGGCU |
| hsa-miR-155 | UUAAUGCUAAUCGUGAUAGGGG |
| hsa-miR-150 | UCUCCCAACCCUUGUACCAGUG |
| hsa-miR-181 | |
| hsa-miR-181a | AACAUUCAACGCUGUCGGUGAGU |
| hsa-miR-181b | AACAUUCAUUGCUGUCGGUGGG |
| hsa-miR-181c | AACAUUCAACCUGUCGGUGAGU |
| hsa-miR-181d | AACAUUCAUUGUUGUCGGUGGGUU |
| hsa-miR-191 | CAACGGAAUCCCAAAAGCAGCU |
| hsa-miR-195 | UAGCAGCACAGAAAUAUUGGC |
| hsa-miR-200c | UAAUACUGCCGGGUAAUGAUGG |
| hsa-miR-197 | UUCACCACCUUCUCCACCCAGC |
| hsa-miR-203 | GUGAAAUGUUUAGGACCACUAG |
| hsa-miR-222 | AGCUACAUCUGGCUACUGGGUCUC |
| hsa-miR-223 | UGUCAGUUUGUCAAAUACCCC |
| hsa-miR-320 | AAAAGCUGGGUUGAGAGGGCGAA |
| hsa-miR-342 | UCUCACACAGAAAUCGCACCCGUC |
| hsa-miR-375 | UUUGUUCGUUCGGCUCGCGUGA |

The miRNA can also be an miRNA which is differentially expressed in squamous cell carcinoma of the tongue. For example, the miRNA can be one found to have an increased level over controls and be selected from the group consisting of hsa-miR-184; hsa-miR-34c; hsa-miR-137; hsa-miR-372; hsa-miR-124a; hsa-miR-21; hsa-miR-124b; hsa-miR-31; hsa-miR-128a; hsa-miR-34b; hsa-miR-154; hsa-miR-197; hsa-miR-132; hsa-miR-147; hsa-miR-325; hsa-miR-181c; hsa-miR-198; hsa-miR-155; hsa-miR-30a-3p; hsa-miR-338; hsa-miR-17-5p; hsa-miR-104; hsa-miR-134; hsa-miR-213. Alternatively, the differentially expressed miRNA can be one with a decreased expression over controls and be selected from the group consisting of hsa-miR-133a; hsa-miR-99a; hsa-miR-194; hsa-miR-133; hsa-miR-219; hsa-miR-100; hsa-miR-125; hsa-miR-26b; hsa-miR-138; hsa-miR-149; hsa-miR-195; hsa-miR-107; and hsa-miR-139.

“Oral cancers” are a part of a group of cancers called head and neck cancers. An oral cancer can develop in any part of the oral cavity or oropharynx. Most oral cancers begin in the tongue and in the floor of the mouth. Almost all oral cancers begin in the flat cells (squamous cells) that cover the surfaces of the mouth, tongue, and lips. These cancers are called oral squamous cell carcinomas (OSCC). When oral cancer cells metastasize, they usually travel through the lymphatic system, carried along by the clear, watery lymphic fluid to secondary sites where they continue to proliferate.

“Therapeutic treatment” and “cancer therapies” refers to chemotherapy, hormonal therapy, radiotherapy, and immunotherapy.

As used in this application, an “increase” or a “decrease” or “differential expression” refers to a detectable positive or negative change in quantity from an established standard control. An increase is a positive change preferably at least 10%, more preferably 50%, still more preferably 2-fold, even more preferably at least 5-fold, and most preferably at least 10-fold of the control value. Similarly, a decrease is a negative change preferably at least 10%, more preferably 50%, still more preferably at least 80%, and most preferably at least 90% of the control. Other terms indicating quantitative changes or differences from a comparative basis, such as “more” or “less,” are used in this application in the same fashion as described above.

“Primers” as used herein refer to oligonucleotides that can be used in an amplification method, such as a polymerase chain reaction (PCR), to amplify a nucleotide sequence based on the polynucleotide sequence corresponding to a sequence of interest, e.g., any miRNA in Table 2, based on the Watson-Crick base-pair complementarity principle.

“Standard control value” as used herein refers to a predetermined amount of a particular miRNA that is detectable in a saliva sample, either in whole saliva or in saliva supernatant. A saliva supernatant can be obtained by centrifugation of saliva (e.g., 5,000 g for 10 minutes at 4° C.)) to separate the cells from the remainder of the fluid. The standard control value is suitable for the use of a method of the present invention, in order for comparing the amount of an miRNA of interest that is present in a saliva sample. An established sample serving as a standard control provides an average amount of the miRNA of interest in the saliva that is typical for an average, healthy person of reasonably matched background, e.g., gender, age, ethnicity, and medical history. A standard control value may vary depending on the miRNA of interest and the nature of the sample (e.g., whole saliva or supernatant).

“Nucleic acid” refers to deoxyribonucleotides or ribonucleotides and polymers thereof in either single- or double-stranded form, and complements thereof. The term encompasses nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphorothioates, phosphoramidates, methyl phosphonates, chiral-methyl phosphonates, 2-O-methyl ribonucleotides, peptide-nucleic acids (PNAs).

Unless otherwise indicated, a particular nucleic acid sequence also implicitly encompasses conservatively modified variants thereof (e.g., degenerate codon substitutions) and complementary sequences, as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); Rossolini et al., Mol. Cell Probes 8:91-98 (1994)). The term nucleic acid is used interchangeably with gene, cDNA, mRNA, oligonucleotide, and polynucleotide.

A particular nucleic acid sequence also implicitly encompasses the particular sequence and “splice variants” and nucleic acid sequences encoding truncated forms of cancer antigens. Similarly, a particular protein encoded by a nucleic acid implicitly encompasses any protein encoded by a splice variant or truncated form of that nucleic acid. “Splice variants,” as the name suggests, are products of alternative splicing of a gene. After transcription, an initial nucleic acid transcript may be spliced such that different (alternate) nucleic acid splice products encode different polypeptides. Mechanisms for the production of splice variants vary, but include alternate splicing of exons. Alternate polypeptides derived from the same nucleic acid by read-through transcription are also encompassed by this definition. Any products of a splicing reaction, including recombinant forms of the splice products, are included in this definition. Nucleic acids can be truncated at the 5′ end or at the 3′ end. Polypeptides can be truncated at the N-terminal end or the C-terminal end. Truncated versions of nucleic acid or polypeptide sequences can be naturally occurring or recombinantly created.

The terms “polypeptide,” “peptide” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymer.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, y-carboxyglutamate, and O-phosphoserine. Amino acid analogs refers to compounds that have the same basic chemical structure as a naturally occurring amino acid, i.e., an α carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

“Conservatively modified variants” applies to both amino acid and nucleic acid sequences. With respect to particular nucleic acid sequences, conservatively modified variants refers to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence with respect to the expression product, but not with respect to actual probe sequences.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the invention.

The following eight groups each contain amino acids that are conservative substitutions for one another: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine (S), Threonine (T); and 8) Cysteine (C), Methionine (M) (see, e.g., Creighton, Proteins (1984)).

The terms “identical” or percent “identity,” in the context of two or more nucleic acids or polypeptide sequences, refer to two or more sequences or subsequences that are the same or have a specified percentage of amino acid residues or nucleotides that are the same (i.e., about 60% identity, preferably 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithms with default parameters described below, or by manual alignment and visual inspection (see, e.g., NCBI web site ncbi.nlm.nih.gov/BLAST/ or the like). Such sequences are then said to be “substantially identical.” This definition also refers to, or may be applied to, the compliment of a test sequence. The definition also includes sequences that have deletions and/or additions, as well as those that have substitutions. As described below, the preferred algorithms can account for gaps and the like. Preferably, identity exists over a region that is at least about 25 amino acids or nucleotides in length, or more preferably over a region that is 50-100 amino acids or nucleotides in length. With respect to miRNA, preferably, identity (90%, 95% or 100%) exists to an miRNA sequence referenced herein over its full length or over a region that is at least 16, 18, 20, or 22 nucleotides in length.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Preferably, default program parameters can be used, or alternative parameters can be designated. The sequence comparison algorithm then calculates the percent sequence identities for the test sequences relative to the reference sequence, based on the program parameters.

A “comparison window”, as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are well-known in the art. Optimal alignment of sequences for comparison can be conducted, e.g., by the local homology algorithm of Smith & Waterman, Adv. Appl. Math. 2:482 (1981), by the homology alignment algorithm of Needleman & Wunsch, J Mol Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Current Protocols in Molecular Biology (Ausubel et al., eds. 1987-2005, Wiley Interscience)).

A preferred example of algorithm that is suitable for determining percent sequence identity and sequence similarity are the BLAST and BLAST 2.0 algorithms, which are described in Altschul et al., Nuc. Acids Res. 25:3389-3402 (1977) and Altschul et al. J Mol. Biol. 215:403-410 (1990), respectively. BLAST and BLAST 2.0 are used, with the parameters described herein, to determine percent sequence identity for the nucleic acids and proteins of the invention. Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al. supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, M=5, N=−4 and a comparison of both strands. For amino acid sequences, the BLASTP program uses as defaults a wordlength of 3, and expectation (E) of 10, and the BLOSUM62 scoring matrix (see Henikoff & Henikoff, Proc. Natl. Acad. Sci. USA 89:10915 (1989)) alignments (B) of 50, expectation (E) of 10, M=5, N=−4, and a comparison of both strands.

A “label” or a “detectable moiety” is a composition detectable by spectroscopic, photochemical, biochemical, immunochemical, chemical, or other physical means. For example, useful labels include ³²P, fluorescent dyes, electron-dense reagents, enzymes (e.g., as commonly used in an ELISA), biotin, digoxigenin, or haptens and proteins which can be made detectable, e.g., by incorporating a radiolabel into the peptide or used to detect antibodies specifically reactive with the peptide.

The term “recombinant” when used with reference, e.g., to a cell, or nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified. Thus, for example, recombinant cells express genes that are not found within the native (non-recombinant) form of the cell or express native genes that are otherwise abnormally expressed, under expressed or not expressed at all.

The phrase “stringent hybridization conditions” refers to conditions under which a probe will hybridize to its target subsequence, typically in a complex mixture of nucleic acids, but to no other sequences. Stringent conditions are sequence-dependent and will be different in different circumstances. Longer sequences hybridize specifically at higher temperatures. An extensive guide to the hybridization of nucleic acids is found in Tijssen, Techniques in Biochemistry and Molecular Biology—Hybridization with Nucleic Probes, “Overview of principles of hybridization and the strategy of nucleic acid assays” (1993). Generally, stringent conditions are selected to be about 5-10° C. lower than the thermal melting point (Tₘ) for the specific sequence at a defined ionic strength pH. The Tₘ is the temperature (under defined ionic strength, pH, and nucleic concentration) at which 50% of the probes complementary to the target hybridize to the target sequence at equilibrium (as the target sequences are present in excess, at Tₘ, 50% of the probes are occupied at equilibrium). Stringent conditions may also be achieved with the addition of destabilizing agents such as formamide. For selective or specific hybridization, a positive signal is at least two times background, preferably 10 times background hybridization. Exemplary stringent hybridization conditions can be as following: 50% formamide, 5×SSC, and 1% SDS, incubating at 42° C., or, 5×SSC, 1% SDS, incubating at 65° C., with wash in 0.2×SSC, and 0.1% SDS at 65° C.

Nucleic acids that do not hybridize to each other under stringent conditions are still substantially identical if the polypeptides which they encode are substantially identical. This occurs, for example, when a copy of a nucleic acid is created using the maximum codon degeneracy permitted by the genetic code. In such cases, the nucleic acids typically hybridize under moderately stringent hybridization conditions. Exemplary “moderately stringent hybridization conditions” include a hybridization in a buffer of 40% formamide, 1 M NaCl, 1% SDS at 37° C., and a wash in 1×SSC at 45° C. A positive hybridization is at least twice background. Those of ordinary skill will readily recognize that alternative hybridization and wash conditions can be utilized to provide conditions of similar stringency. Additional guidelines for determining hybridization parameters are provided in numerous reference, e.g., and Current Protocols in Molecular Biology, ed. Ausubel, et al., supra.

For PCR, a temperature of about 36° C. is typical for low stringency amplification, although annealing temperatures may vary between about 32° C. and 48° C. depending on primer length. For high stringency PCR amplification, a temperature of about 62° C. is typical, although high stringency annealing temperatures can range from about 50° C. to about 65° C., depending on the primer length and specificity. Typical cycle conditions for both high and low stringency amplifications include a denaturation phase of 90° C.-95° C. for 30 sec-2 min., an annealing phase lasting 30 sec.-2 min., and an extension phase of about 72° C. for 1-2 min. Protocols and guidelines for low and high stringency amplification reactions are provided, e.g., in Innis et al. (1990) PCR Protocols, A Guide to Methods and Applications, Academic Press, Inc. N.Y.).

“Antibody” refers to a polypeptide comprising a framework region from an immunoglobulin gene or fragments thereof that specifically binds and recognizes an antigen. The recognized immunoglobulin genes include the kappa, lambda, alpha, gamma, delta, epsilon, and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively. Typically, the antigen-binding region of an antibody will be most critical in specificity and affinity of binding.

An exemplary immunoglobulin (antibody) structural unit comprises a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms variable light chain (V_{L}) and variable heavy chain (V_{H}) refer to these light and heavy chains respectively.

Antibodies exist, e.g., as intact immunoglobulins or as a number of well-characterized fragments produced by digestion with various peptidases. Thus, for example, pepsin digests an antibody below the disulfide linkages in the hinge region to produce F(ab)′₂, a dimer of Fab which itself is a light chain joined to V_{H}-C^{H}1 by a disulfide bond. The F(ab)′₂ may be reduced under mild conditions to break the disulfide linkage in the hinge region, thereby converting the F(ab)′₂ dimer into an Fab′ monomer. The Fab′ monomer is essentially Fab with part of the hinge region (see Fundamental Immunology (Paul ed., 3d ed. 1993). While various antibody fragments are defined in terms of the digestion of an intact antibody, one of skill will appreciate that such fragments may be synthesized de novo either chemically or by using recombinant DNA methodology. Thus, the term antibody, as used herein, also includes antibody fragments either produced by the modification of whole antibodies, or those synthesized de novo using recombinant DNA methodologies (e.g., single chain Fv) or those identified using phage display libraries (see, e.g., McCafferty et al., Nature 348:552-554 (1990))

For preparation of antibodies, e.g., recombinant, monoclonal, or polyclonal antibodies, many technique known in the art can be used (see, e.g., Kohler & Milstein, Nature 256:495-497 (1975); Kozbor et al., Immunology Today 4: 72 (1983); Cole et al., pp. 77-96 in Monoclonal Antibodies and Cancer Therapy, Alan R. Liss, Inc. (1985); Coligan, Current Protocols in Immunology (1991); Harlow & Lane, Antibodies, A Laboratory Manual (1988); and Goding, Monoclonal Antibodies: Principles and Practice (2d ed. 1986)). The genes encoding the heavy and light chains of an antibody of interest can be cloned from a cell, e.g., the genes encoding a monoclonal antibody can be cloned from a hybridoma and used to produce a recombinant monoclonal antibody. Gene libraries encoding heavy and light chains of monoclonal antibodies can also be made from hybridoma or plasma cells. Random combinations of the heavy and light chain gene products generate a large pool of antibodies with different antigenic specificity (see, e.g., Kuby, Immunology (3^{rd} ed. 1997)). Techniques for the production of single chain antibodies or recombinant antibodies (U.S. Pat. No. 4,946,778, U.S. Pat. No. 4,816,567) can be adapted to produce antibodies to polypeptides of this invention. Also, transgenic mice, or other organisms such as other mammals, may be used to express humanized or human antibodies (see, e.g., U.S. Pat. Nos. 5,545,807; 5,545,806; 5,569,825; 5,625,126; 5,633,425; 5,661,016, Marks et al., Bio/Technology 10:779-783 (1992); Lonberg et al., Nature 368:856-859 (1994); Morrison, Nature 368:812-13 (1994); Fishwild et al., Nature Biotechnology 14:845-51 (1996); Neuberger, Nature Biotechnology 14:826 (1996); and Lonberg & Huszar, Intern. Rev. Immunol. 13:65-93 (1995)). Alternatively, phage display technology can be used to identify antibodies and heteromeric Fab fragments that specifically bind to selected antigens (see, e.g. McCafferty et al. Nature 348:552-554 (1990); Marks et al. Biotechnology 10:779-783 (1992)). Antibodies can also be made bispecific, i.e., able to recognize two different antigens (see, e.g., WO 93/08829, Traunecker et al., EMBO J. 10:3655-3659 (1991); and Suresh et al., Methods in Enzymology 121:210 (1986)). Antibodies can also be heteroconjugates, e.g., two covalently joined antibodies, or immunotoxins (see, e.g., U.S. Pat. No. 4,676,980, WO 91/00360; WO 92/200373; and EP 03089).

Methods for humanizing or primatizing non-human antibodies are well known in the art. Generally, a humanized antibody has one or more amino acid residues introduced into it from a source which is non-human. These non-human amino acid residues are often referred to as import residues, which are typically taken from an import variable domain. Humanization can be essentially performed following the method of Winter and co-workers (see, e.g., Jones et al., Nature 321:522-525 (1986); Riechmann et al., Nature 332:323-327 (1988); Verhoeyen et al., Science 239:1534-1536 (1988) and Presta, Curr. Op. Struct. Biol. 2:593-596 (1992)), by substituting rodent CDRs or CDR sequences for the corresponding sequences of a human antibody. Accordingly, such humanized antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

A “chimeric antibody” is an antibody molecule in which (a) the constant region, or a portion thereof, is altered, replaced or exchanged so that the antigen binding site (variable region) is linked to a constant region of a different or altered class, effector function and/or species, or an entirely different molecule which confers new properties to the chimeric antibody, e.g., an enzyme, toxin, hormone, growth factor, drug, etc.; or (b) the variable region, or a portion thereof, is altered, replaced or exchanged with a variable region having a different or altered antigen specificity.

In one embodiment, the antibody is conjugated to an “effector” moiety. The effector moiety can be any number of molecules, including labeling moieties such as radioactive labels or fluorescent labels, or can be a therapeutic moiety. In one aspect the antibody modulates the activity of the protein.

The phrase “specifically (or selectively) binds” to an antibody or “specifically (or selectively) immunoreactive with,” when referring to a protein or peptide, refers to a binding reaction that is determinative of the presence of the protein, often in a heterogeneous population of proteins and other biologics. Thus, under designated immunoassay conditions, the specified antibodies bind to a particular protein at least two times the background and more typically more than 10 to 100 times background. Specific binding to an antibody under such conditions requires an antibody that is selected for its specificity for a particular protein. For example, polyclonal antibodies can be selected to obtain only those polyclonal antibodies that are specifically immunoreactive with the selected antigen and not with other proteins. This selection may be achieved by subtracting out antibodies that cross-react with other molecules. A variety of immunoassay formats may be used to select antibodies specifically immunoreactive with a particular protein. For example, solid-phase ELISA immunoassays are routinely used to select antibodies specifically immunoreactive with a protein (see, e.g., Harlow & Lane, Antibodies, A Laboratory Manual (1988) for a description of immunoassay formats and conditions that can be used to determine specific immunoreactivity).

### Diagnostic and Prognostic Methods

The present invention provides methods of diagnosing an oral cancer by examining relevant miRNA species in saliva samples, including detecting quantitative changes in miRNA levels compared with a control. Diagnosis involves determining the level of one or more miRNA of the invention in a patient's saliva sample and then comparing the level to a baseline or range. Typically, the baseline value is representative of an miRNA of the invention in a healthy person not suffering from cancer, as measured using saliva samples processed in the same manner. Variation of levels of an miRNA of the invention from the baseline range (either up or down) indicates that the patient has an oral cancer or is at risk of developing an oral cancer.

As used herein, the term “providing a prognosis” refers to providing a prediction of the probable course and outcome of an oral cancer such as OSCC, including prediction of metastasis, disease free survival, overall survival, etc. The methods can also be used to devise a suitable therapy for cancer treatment, e.g., by indicating whether or not the cancer is still at an early stage or if the cancer had advanced to a stage where aggressive therapy would be ineffective.

Nucleic acid binding molecules such as probes, oligonucleotides, oligonucleotide arrays, and primers can be used in assays to detect differential expression of miRNA relevant to oral cancer, e.g., RT-PCR. In one embodiment, RT-PCR is used according to standard methods known in the art. In another embodiment, PCR assays such as Taqman® assays available from, e.g., Applied Biosystems, can be used to detect nucleic acids and variants thereof. In other embodiments, qPCR and nucleic acid microarrays can be used to detect nucleic acids. Reagents that bind to selected cancer biomarkers can be prepared according to methods known to those of skill in the art or purchased commercially.

Analysis of nucleic acids can be achieved using routine techniques such as Southern analysis, reverse-transcriptase polymerase chain reaction (RT-PCR), or any other methods based on hybridization to a nucleic acid sequence that is complementary to a portion of the marker coding sequence (e.g., slot blot hybridization) are also within the scope of the present invention. Applicable PCR amplification techniques are described in, e.g., Ausubel et al. and Innis et al., supra. General nucleic acid hybridization methods are described in Anderson, “Nucleic Acid Hybridization,” BIOS Scientific Publishers, 1999. Amplification or hybridization of a plurality of nucleic acid sequences (e.g., genomic DNA, mRNA or cDNA) can also be performed from mRNA or cDNA sequences arranged in a microarray. Microarray methods are generally described in Hardiman, “Microarrays Methods and Applications: Nuts & Bolts,” DNA Press, 2003; and Baldi et al., “DNA Microarrays and Gene Expression From Experiments to Data Analysis and Modeling,” Cambridge University Press, 2002.

Analysis of miRNA markers can be performed using techniques known in the art including, without limitation, microarrays, polymerase chain reaction (PCR)-based analysis, sequence analysis, and electrophoretic analysis. A non-limiting example of a PCR-based analysis includes a Taqman® allelic discrimination assay available from Applied Biosystems. Non-limiting examples of sequence analysis include Maxam-Gilbert sequencing, Sanger sequencing, capillary array DNA sequencing, thermal cycle sequencing (Sears et al., Biotechniques, 13:626-633 (1992)), solid-phase sequencing (Zimmerman et al., Methods Mol. Cell Biol., 3:39-42 (1992)), sequencing with mass spectrometry such as matrix-assisted laser desorption/ionization time-of-flight mass spectrometry (MALDI-TOF/MS; Fu et al., Nat. Biotechnol., 16:381-384 (1998)), and sequencing by hybridization. Chee et al., Science, 274:610-614 (1996); Drmanac et al., Science, 260:1649-1652 (1993); Drmanac et al., Nat. Biotechnol., 16:54-58 (1998). Non-limiting examples of electrophoretic analysis include slab gel electrophoresis such as agarose or polyacrylamide gel electrophoresis, capillary electrophoresis, and denaturing gradient gel electrophoresis.

A detectable moiety can be used in the assays described herein. A wide variety of detectable moieties can be used, with the choice of label depending on the sensitivity required, ease of conjugation with the antibody, stability requirements, and available instrumentation and disposal provisions. Suitable detectable moieties include, but are not limited to, radionuclides, fluorescent dyes (e.g., fluorescein, fluorescein isothiocyanate (FITC), Oregon Green™, rhodamine, Texas red, tetrarhodimine isothiocynate (TRITC), Cy3, Cy5, etc.), fluorescent markers (e.g., green fluorescent protein (GFP), phycoerythrin, etc.), autoquenched fluorescent compounds that are activated by tumor-associated proteases, enzymes (e.g., luciferase, horseradish peroxidase, alkaline phosphatase, etc.), nanoparticles, biotin, digoxigenin, and the like.

Useful physical formats comprise surfaces having a plurality of discrete, addressable locations for the detection of a plurality of different miRNA markers. Such formats include microarrays and certain capillary devices. See, e.g., Ng et al., J Cell Mol. Med., 6:329-340 (2002); U.S. Pat. No. 6,019,944. In these embodiments, each discrete surface location may comprise antibodies to immobilize one or more markers for detection at each location. Surfaces may alternatively comprise one or more discrete particles (e.g., microparticles or nanoparticles) immobilized at discrete locations of a surface, where the microparticles comprise antibodies to immobilize one or more markers for detection. Other useful physical formats include sticks, wells, sponges, and the like.

Analysis can be carried out in a variety of physical formats. For example, the use of microtiter plates or automation could be used to facilitate the processing of large numbers of test samples. Alternatively, single sample formats could be developed to facilitate diagnosis or prognosis in a timely fashion.

Alternatively, the nucleic acid probes of the invention can be applied to patient samples immobilized on microscope slides. The resulting in situ hybridization pattern can be visualized using any one of a variety of light or fluorescent microscopic methods known in the art.

Analysis of the miRNA markers can also be achieved, for example, by high pressure liquid chromatography (HPLC), alone or in combination with mass spectrometry (e.g., MALDI/MS, MALDI-TOF/MS, tandem MS, etc.).

In some embodiments of the invention in any of its various aspects, the disease state is squamous cell carcinoma of the tongue or oral cavity and the detected miRNA which is associated with the cancer is selected from the following list of miRNAs reported to be up-regulated (fold changes from matched controls are provided) in laser microdissected cells from squamous cell carcinomas (SCC) of the tongue:

| | Fold | |
|---|---|---|
| Mature microRNAs | changes | Sequence |
| hsa-miR-184 | 59 | uggacggagaacugauaagggu |
| hsa-miR-34c | 57 | aggcaguguaguuagcugauug |
| hsa-miR-137 | 30 | uauugcuuaagaauacgcguag |
| hsa-miR-372 | 26 | aaagugcugcgacauuugagcgu |
| hsa-miR-124a | 14 | uuaaggcacgcggugaaugcca |
| hsa-miR-21 | 12 | uagcuuaucagacugauguuga |
| hsa-miR-124b | 10 | uuaaggcacgcggugaaugc |
| hsa-miR-31 | 6 | ggcaagaugcuggcauagcug |
| hsa-miR-128a | 5 | ucacagugaaccggucucuuuu |
| hsa-miR-34b | 5 | aggcagugucauuagcugauug |
| hsa-miR-154 | 5 | aaucauacacgguugaccuauu |
| hsa-miR-197 | 4 | uucaccaccuucuccacccagc |
| hsa-miR-132 | 4 | uaacagucuacagccauggucg |
| hsa-miR-147 | 4 | guguguggaaaugcuucugc |
| hsa-miR-325 | 4 | ccuaguagguguccaguaagu |
| hsa-miR-181c | 3 | aacauucaaccugucggugagu |
| hsa-miR-198 | 3 | gguccagaggggagauagg |
| hsa-miR-155 | 3 | uuaaugcuaaucgugauagggg |
| hsa-miR-30a-3p | 3 | cuuucagucggauguuugcagc |
| hsa-miR-338 | 3 | acauagaggaaauuccacguuu |
| hsa-miR-17-5p | 3 | caaagugcuuacagugcagguagu |
| hsa-miR-104 | 3 | ucaacaucagucugauaagcua |
| hsa-miR-134 | 3 | ugugacugguugaccagaggg |
| hsa-miR-213 | 3 | accaucgaccguugauuguacc |

(see, Wong et al., Clin Cancer Res. 2008 May 1; 14(9):2588-92).

In some embodiments, the miRNA is hsa-miR-184, hsa-miR-34c, hsa-miR-137, hsa-miR-372, hsa-miR-124a, hsa-miR-21, or hsa-miR-124b.

In other embodiments of the invention in any of its various aspects, the disease state is squamous cell carcinoma of the tongue or oral cavity and the detected miRNA which is associated with the cancer is selected from the following list of miRNAs down-regulated in SCC of tongue:

| | Fold | |
|---|---|---|
| Mature microRNAs | change | Sequence |
| hsa-miR-133a | −13 | uugguccccuucaaccagcugu |
| hsa-miR-99a | −9 | aacccguagauccgaucuugug |
| hsa-miR-194 | −6 | uguaacagcaacuccaugugga |
| hsa-miR-133b | −5 | uugguccccuucaaccagcua |
| hsa-miR-219 | −5 | ugauuguccaaacgcaauucu |
| hsa-miR-100 | −5 | aacccguagauccgaacuugug |
| hsa-miR-125b | −5 | ucccugagacccuaacuuguga |
| hsa-miR-26b | −4 | uucaaguaauucaggauaggu |
| hsa-miR-138 | −4 | agcugguguugugaauc |
| hsa-miR-149 | −4 | ucuggcuccgugucuucacucc |
| hsa-miR-195 | −3 | uagcagcacagaaauauuggc |
| hsa-miR-107 | −3 | agcagcauuguacagggcuauca |
| hsa-miR-139 | −3 | ucuacagugcacgugucu |

see, Wong et al., Clin Cancer Res. 2008 May 1; 14(9):2588-92).

### Compositions, Kits and Integrated Systems

The invention provides compositions, kits and integrated systems for practicing the assays described herein using nucleic acids specific for the miRNA polynucleotide sequences of the invention.

Kits for carrying out the diagnostic assays of the invention typically include a probe that comprises a nucleic acid sequence that specifically binds to miRNA sequences of this invention, and a label for detecting the presence of the probe. The kits may include several polynucleotide probes for hybridizing with miRNA of this invention, e.g., a cocktail of probes that recognize miR-200a, miR-125a, miR-142-3p, and miR-93. The kits may also contain a container for the saliva sample, as well as an inhibitor of salivary RNAse activity and optionally devices (e.g., swab, scrappers) to collect a saliva sample from the subject. In some embodiments, the inhibitor can be RNA or RNAprotect©Saliva Reagent (RPS, Qiagen Inc., Valencia Calif.) (see, Jiang et al., Arch. Oral Biology 54(3):268-273 (1999)). The kit can be used in any setting where sample collectin and RNA preservation in saliva is desired (e.g., pediatrician's, family doctor's, dentist's, other health care providers' offices, community clinics, home-care kits). The preserved RNA can then be shipped to a diagnostic center for specific RNA-based screening or diagnostics. We envision kits for collecting saliva, such as, for example, described in U.S. Pat. Nos. 6,652,481; 6,022,326; 5,393,496; 5,910,122; 5,376,337; 4,019,255; and 4,768,238. In some embodiments, RNAlater™-type RNAse inhibiting composition is the inhibitor.

### EXAMPLES

The following examples are offered to illustrate, but not to limit the claimed invention.

### Introduction

It has been previously reported that mRNAs are found in saliva and they can be used as oral cancer biomarkers. In this study, the present inventors measured the presence of micro-RNA (miRNA) in saliva and explored the utility of miRNA as additional oral cancer biomarkers. A total of 314 miRNAs were measured using reverse transcriptase-preamplification-quantitativePCR (RT-preamp-qPCR) in 12 healthy subjects. Degradation of endogenous and exogenous saliva miRNAs were measured at room temperature for different periods of time. Selected miRNAs were compared in saliva of 50 oral squamous cell carcinoma (OSCC) and 50 gender, age, and ethnicity-matching healthy subjects. On average, about 50 miRNAs were detected in both whole and supernatant saliva. Endogenous saliva miRNA level decreased much slower compared to the exongenous miRNA. Two miRNAs, miR-125a and miR-200a, are present in significantly different levels (p<0.05) between OSCC and control groups. Both the whole and supernatant saliva of healthy subjects contain dozens of miRNAs, and just like saliva mRNA, miRNA detected in saliva also appear to be stable. Saliva miRNA thus can be used for oral cancer detection.

### Materials and Methods

### Saliva Samples

Whole saliva samples were preserved with RNAlater (QIAGEN Inc., Valencia, Calif.), and supernatant saliva samples were preserved with SUPERaseIn™ (Ambion Inc., Austin, Tex.) as described previously (28). All of the saliva samples were kept at −80° C. at all times. All of the volunteers signed the UCLA institutional review board-approved consent for participating in the study. The average age of OSCC volunteers was 56 and they were consisted of 41 Caucasians and 4 Asians, 4 Hispanics, and 1 African American; 32 males and 18 females. The average age of control volunteers was 52 and they were consisted of 39 Caucasians and 3 Asians, 3 Hispanics, and 5 African American; 29 males and 21 females. The volunteers had no history of malignancy, immunodeficiency, autoimmune disorders, hepatitis or HIV infection. The cancer stage of OSCC volunteers ranged from stages I to IV.

### Saliva RNA Extraction

400 μL of whole saliva mixture (200 μL whole saliva and 200 μL RNAlater), and 400 μL of supernatant saliva were used for RNA extraction. Saliva samples were extracted using mirVana™ miRNA Isolation Kit according to the manufacturer's guideline (Ambion Inc., Austin, Tex.). For the initial lysis step, per 400 μL saliva sample, we used 1 mL of Lysis/Binding solution. After the extraction, 100 μL of purified RNA was digested with DNA-free™ (Ambion Inc.) to completely remove any genomic DNA. Then, the RNA samples were concentrated to 20 μL using Vacufuge (Eppendorf, Westbury, N.Y.).

### RT-preamp-qPCR of 12 Healthy Subjects

We analyzed a total of 314 miRNAs, and all the reagents used for RT-preamp-qPCR are from Applied Biosystems (Foster City, Calif.). RT and preamp were carried out using PTC-200 thermal cycler from Bio-Rad Laboratories (Hercules, Calif.), and qPCR reactions were performed using 7500 and 7900HT Fast Real-Time PCR systems (Applied Biosystems, Foster City, Calif.).

For 314 and 71-plex RT-preamp, total of 5 μL RT reaction contains following: 2 μL RNA, 0.5 μL 10×RT primer mix (314 miRNA multiplex), 0.1 μL 25 mM dNTPs, 1 μL 50U/μL MultiScribe Reverse Transcriptase, 0.5 μL 10×RT buffer, 0.6 μL 25 mM MgCl2, 0.06 μL 20U/μL AB RNase Inhibitor, and 0.24 μL water. The RT reaction was carried out as following: (16° C. for 2 min, 42° C. for 1 min, and 50° C. for 1 sec) for 40 cycles and 85° C. for 5 min. Preamplification reaction contains 5 μL of RT, 12.5 μL 5× Preamp Primer Mix, 5 μL 314 multiplex 5× preamp primer mix (250 mM each), and 2.5 μL water. The preamplification reaction was carried out as following: 95° C. for 10 min, 55° C. for 2 min, 72° C. for 2 min, and (95° C. for 15 sec, 60° C. for 4 min) for 14 cycles. Then, the preamp product was diluted 4 fold by adding 75 μL of water. A 10 μL qPCR reaction contains 0.025 μL diluted preamp product, 5 μL 2× TaqMan Master Mix no UNG, 2.975 μL water, and 2 μL 5 × PCR probe/primer mix. All the qPCR reactions were done in duplicates.

### RT-preamp-qPCR in 50 OSCC and 50 Control Subjects

Four-plex RT-preamp-qPCR amplifies four of following miRNAs: miR-142-3p, miR-200a, miR-125a, and miR-93. For RT, instead of using mega-plex RT protocol, we used standard ABI RT reaction condition that contains following: total of 7.5 μL reaction contains 1 μL RNA, 0.075 μL dNTP mix, 0.5 μL 50U/μL MultiScribe Reverse Transcriptase, 0.75 μL 10×RT buffer, 0.095 μL AB RNase Inhibitor, 3.58 μL water, and 1.5 μL that contains 0.375 μL each of 4 primers. RT reaction was carried out at 16° C. for 30 min and 42° C. for 30 min. Preamp was done as described above. Preamp product was diluted 4 fold with water, and 0.1 μL of cDNA was used for qPCR as described above.

Saliva miRNA Stability Assay

To 10 mL of pooled supernatant saliva, 5 μL of 100 μM miR-124a was added, and 400 μL of triplicate samples were removed at each time point and immediately incubated with Lysis/Binding Solution, a component of mirVana™ miRNA Isolation Kit, until the time course was completed. Extracted RNA was digested with DNA-free™, and concentrated to 30 μL. Two μL of purified RNA was used for RT as described above. RT was then diluted 10 fold with water, and 2 μL of diluted RT product was used for 10 μL qPCR reaction as described above. qPCR was done in duplicates.

### Results

miRNAs in saliva—Our previous results showed that thousands of mRNAs can be found in the supernatant saliva, and some of these mRNAs can be used for oral cancer detection (5-7). To further investigate the presence of potential disease markers in saliva, we tested the presence of miRNAs in saliva. Both whole and supernatant saliva from 12 healthy subjects were used for miRNA profiling. We initially measured 314 miRNAs from 6 subjects using RT-preamp-qPCR. We arbitrarily considered miRNAs with CT value lower than 35 as present in saliva. Of 314 miRNAs we have initially analyzed, we found 71 miRNAs to be present in at least 2 subjects. We then further analyzed these 71 miRNAs in the second set of 6 samples. Our results from these 12 subjects indicate that on average, we detected 47 miRNAs in 200 μL of the whole saliva and 52 miRNAs in 400 of the supernatant saliva (Table 1):

TABLE 1
Number of miRNAs present in saliva

| Subject | Whole | Super |
|---|---|---|
| 1 | 19 | 46 |
| 2 | 22 | 37 |
| 3 | 9 | 17 |
| 4 | 52 | 58 |
| 5 | 62 | 55 |
| 6 | 60 | 53 |
| 7 | 65 | 66 |
| 8 | 47 | 58 |
| 9 | 64 | 66 |
| 10 | 45 | 50 |
| 11 | 63 | 56 |
| 12 | 62 | 60 |
| Average | 47 | 52 |
| Stdev | 20 | 14 |
| % | 14.90% | 16.60%. |

In the whole saliva, 13 of these 47 miRNAs were present in at least 11 of 12 subjects, and in the supernatant saliva, 28 of 52 miRNAs were present in at least 11 of 12 subjects (Table 2):

TABLE 2
miRNA list that are frequently found in saliva

| Whole saliva | Supernatant saliva | |
|---|---|---|
| Hsa-mir-16 | Hsa-mir-16 | Let-7b |
| Hsa-mir-19b | Hsa-mir-19b | Hsa-mir-26a |
| Hsa-mir-24 | Hsa-mir-24 | Hsa-mir-30c |
| Hsa-mir-26b | Hsa-mir-26b | Hsa-mir-30a-3p |
| Hsa-mir-30e-3p | Hsa-mir-30e-3p | Hsa-mir-30e-5p |
| Hsa-mir-92 | Hsa-mir-92 | Hsa-mir-125a |
| Hsa-mir-146a | Hsa-mir-146a | Hsa-mir-140 |
| Hsa-mir-146b | Hsa-mir-146b | Hsa-mir-155 |
| Hsa-mir-150 | Hsa-mir-150 | Hsa-mir-181 |
| Hsa-mir-191 | Hsa-mir-191 | Hsa-mir-195 |
| Hsa-mir-200c | Hsa-mir-200c | Hsa-mir-197 |
| Hsa-mir-203 | Hsa-mir-203 | Hsa-mir-222 |
| Hsa-mir-223 | Hsa-mir-223 | Hsa-mir-320 |
| Hsa-mir-200a | | Hsa-mir-342 |
| Hsa-mir-125a | Hsa-mir-200a | Hsa-mir-375 |
| Hsa-mir-142-3p | Hsa-mir-142-3p | |
| Hsa-mir-93 | Hsa-mir-93 | |

It is surprising that higher number of miRNAs were found in the supernatant saliva compared to the whole saliva. A partial reason for this is due to extracting RNA from 400 μL of supernatant saliva and 200 μL for the whole saliva. In addition, it also could be due to extracted whole saliva RNA containing degraded small non-miRNAs that may reduce the efficiency of some of the steps in the RT-preamp-qPCR. Nonetheless, the whole and supernatant saliva showed remarkable similarity since all of the 13 miRNAs present in the whole saliva are also present in the supernatant saliva. Together our data indicate that both the whole and supernatant saliva contain detectable amount of miRNAs, and there appear to be common miRNAs that are present in healthy subjects.

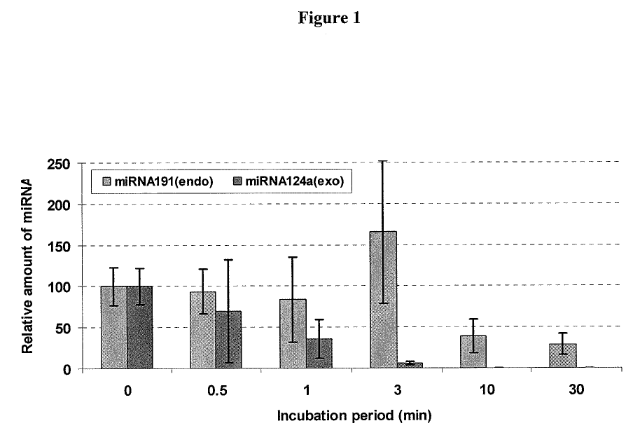

miRNA stability in saliva—We previously showed that saliva mRNAs are partially protected from degradation due to association with unidentified macromolecules (8). Such a mechanism is also observed in plasma and serum (8, 29, 30). To test if miRNAs are also protected from degradation, we measured the degradation pattern of endogenous and exogenous miRNAs. As for the endogenous saliva miRNA, we measured the miR-191, which showed consistently low CT values across all the saliva samples we have tested. As for the exogenous miRNA, we designed an RNA oligo, where its sequence matches to miR-124a. Our data from 12 saliva samples indicated that miR-124a is not present in any of the saliva samples we have tested, thus serves as an exogenous RNA input without endogenous contamination. At time 0, we added exogenous miR-124a to the saliva, and the time course was carried out at room temperature for up to 30 minutes. Saliva aliquots were removed at different time points, and RT-qPCR was performed on the purified total RNAs for miR-191 and miR-124a. FIG. 1 shows that the level of the exogenous miR-124a show a rapid decrease during the time course and by 3 minutes, less than 10% of miR-124a was detected, suggesting that miRNA 124a degrades rapidly in saliva. Endogenous miR-191 on contrary shows much slower decrease in its level, and by 30 minutes, about 30% of miR-191 were detected. Together, these data suggest that endogenous miRNAs are degraded slower than exogenous miRNAs, suggesting that there is a protection mechanism for endogenous miRNAs.

To test if saliva miRNAs can be used for oral cancer detection, we compared saliva miRNA profiles between OSCC and control subjects matched for age, gender, ethnicity and smoking history. Saliva supernatant was analyzed to avoid miRNA contamination from cells. In the initial 12 control and 12 OSCC dataset, four potential miRNA candidate markers were identified to be statistically significance (P<0.05). They are miR-200a, miR-125a, miR-142-3p and miR-93 (Table 2A):

TABLE 2A
Summary of potential OSCC miRNA markers in 12 OSCC and
12 control subjects

| | Median | Median | | | | |
|---|---|---|---|---|---|---|
| | C_{T} in | C_{T} in | OSCC | Control | P | |
| miRNA | OSCC | control | SD | SD | value^{a} | AUC^{a} |
| miR-200a | 35.25 | 34.25 | 2.08 | 3.76 | 0.05 | 0.54 |
| miR-125a | 32.30 | 30.70 | 1.83 | 3.28 | 0.05 | 0.53 |
| miR-93 | 33.30 | 33.06 | 4.02 | 4.19 | 0.04 | 0.52 |
| miR-142-3p | 37.84 | 32.62 | 3.04 | 2.19 | 0.02 | 0.59 |

SD: Standard Deviation
^{a}Both the p value and AUC are obtained using the U6-normalized values. The Mann-Whitney U test was used to obtain the p values.

We then further tested the potential significance of these 4 miRNAs in additional independent cohort of 38 control and 38 OSCC samples using RT-preamp-qPCR. Since we only wished to measure these four 4 miRNAs, we used a simplified RT reaction in this set of experiment (see Materials and Methods). We also repeated the RT-preamp-qPCR on initial 12 control and 12 OSCC subjects using the simplified RT condition (see Materials and Methods). Table 2B shows that the average CT, p, and AUC values of these miRNAs in combined 50 control and 50 OSCC subjects:

TABLE 2B
Summary of potential OSCC miRNA markers in 50 OSCC and
50 control subjects

| | Median | Median | | | | |
|---|---|---|---|---|---|---|
| | CT in | CT in | OSCC | Control | | |
| miRNA | OSCC | control | SD | SD | P value^{a} | AUC^{a} |
| miR-200a | 28.7 | 27.7 | 3.94 | 3.94 | 0.01 | 0.65 |
| miR-125a | 22.8 | 22.4 | 3.28 | 2.85 | 0.03 | 0.62 |
| miR-93 | 20.2 | 20.1 | 3.79 | 3.29 | 0.17 | 0.57 |
| miR-142-3p | 19.6 | 19.2 | 3.28 | 3.11 | 0.18 | 0.58 |

SD: Standard Deviation
^{a}Both the p value and AUC are obtained using the U6-normalized values. The Mann-Whitney U test was used to obtain the p values.

The p-values for miR-200a and miR-125a were significantly different between these two groups; 0.01 and 0.03 respectively. However, the p-values for miR-142-3p and miR-93 were 0.18 and 0.17, respectively, which indicate that these miRNAs are not significantly different between the control and OSCC groups. AUC for miR-200a and miR-125a are 0.65 and 0.62 respectively, whereas the AUC for miR-142-3p and miR-93 were lower; 0.58 and 0.57, respectively. The standard deviation and interquartile range of RT-preamp-PCR results were also included in Table 2. Together, these data suggest that miRNAs miR-200a and miR-125a are present in significantly different levels between the OSCC and control groups.

### Discussions

Saliva is important for food digestion, speech, and defense against microorganisms. Reports from our group showed previously that saliva mRNAs can be used as biomarkers for oral cancer, and combined measurement of 7 different mRNAs showed specificity and sensitivity of 0.91 respectively for oral cancer discrimination (5, 7). To enhance diagnostic power of saliva for oral cancer, we profiled salivary miRNA and measured the utility of miRNAs as diagnostic markers. We have shown that both whole and supernatant saliva contain miRNAs, and their profiles are highly similar. Similar to mRNAs in saliva, our data indicate that saliva miRNAs are stable compared to exogenous miRNA. Comparisons of saliva miRNAs between OSCC and control subjects showed that a panel of miRNAs is present in different amount between these two groups.

We repeatedly detected 13 miRNAs in healthy subjects' whole saliva and 28 for the supernatant saliva. There is remarkable similarity between the whole and supernatant saliva miRNA profiles since all 13 miRNAs in whole saliva were also consistently detected in the supernatant saliva. It is unexpected that RT-preamp-qPCR detected more miRNAs in the supernatant than the whole saliva. It is unlikely that that the supernatant saliva actually contains more miRNAs than the whole saliva. One explanation for detecting less miRNAs in the whole saliva is due to the whole saliva containing analytes that interfere with miRNA amplification during the RT-preamp-qPCR. A possible interfering analyte is degraded small RNAs that are not miRNA origin. During apoptosis, RNA as well as DNA undergoes degradation(31). Therefore, in addition to miRNAs, whole saliva may have fragmented RNA species, which can potentially compete for same substrates with miRNAs during the RT-preamp-qPCR.

Our previous data showed that saliva mRNAs are stable as compared to naked exogenous RNA due to their association with macromolecules(8). In cells, mature miRNAs are bound by RISC complex, and most likely this interaction confers miRNA stability in cells. It is likely that saliva miRNAs are also bound by RISC, and thus confer stability in saliva. Western blotting of saliva proteins against an antibody specific to Ago 2, a component of the RISC complex, showed that Ago 2 is present in supernatant saliva (data not shown).

We found 2 miRNAs, miR-200a and miR-125a, where their expression level is lower in OSCC as compared to the control subjects. Through transient transfection studies, miR-125a along with it homolog miR-125b have been shown to reduce ERBB2 and ERBB3 oncogenic protein levels in a human breast cancer cell line SKBR3 (32). miR-200a has been reported to be differentially expressed in head and neck cancer cell lines and other cancer cells (27, 33-35). Two different reports using both microarray and RT-PCR system showed that miR-200a are present in higher amount in the head and neck cancer cell lines (27, 35). Interestingly, miR-200a is present in lower amount in the OSCC patients compared to the control subjects.

In conclusion, we showed that miRNAs are present in both the whole and supernatant saliva, and two of the miRNAs miR-125a and miR-200a appear to be differentially expressed in saliva of OSCC compared to control subjects. These findings suggest that miRNAs in saliva can be used for oral cancer detection, and combining both the mRNA and miRNA markers may result in diagnostic markers with higher sensitivity and specificity.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

### REFERENCES

1. Brinkman B M, Wong D T. Disease mechanism and biomarkers of oral squamous cell carcinoma. Curr Opin Oncol 2006; 18:228-33.
2. Sano D, Myers J N. Metastasis of squamous cell carcinoma of the oral tongue. Cancer Metastasis Rev 2007; 26:645-62.
3. Funk G F, Karnell L H, Robinson R A, Zhen W K, Trask D K, Hoffman H T. Presentation, treatment, and outcome of oral cavity cancer: a National Cancer Data Base report. Head Neck 2002; 24:165-80.
4. Chai R L, Grandis J R. Advances in molecular diagnostics and therapeutics in head and neck cancer. Curr Treat Options Oncol 2006; 7:3-11.
5. Li Y, St John M A, Zhou X, Kim Y, Sinha U, Jordan R C, et al. Salivary transcriptome diagnostics for oral cancer detection. Clin Cancer Res 2004; 10:8442-50.
6. Li Y, Zhou X, St John M A, Wong D T. RNA profiling of cell-free saliva using microarray technology. J Dent Res 2004; 83:199-203.
7. St John M A, Li Y, Zhou X, Denny P, Ho C M, Montemagno C, et al. Interleukin 6 and interleukin 8 as potential biomarkers for oral cavity and oropharyngeal squamous cell carcinoma. Arch Otolaryngol Head Neck Surg 2004; 130:929-35.
8. Park N J, Li Y, Yu T, Brinkman B M, Wong D T. Characterization of RNA in saliva. Clin Chem 2006; 52:988-94.
9. Park N J, Zhou X, Yu T, Brinkman B M, Zimmermann B G, Palanisamy V, Wong D T. Characterization of salivary RNA by cDNA library analysis. Arch Oral Biol 2006.
10. Grosshans H, Slack F J. Micro-RNAs: small is plentiful. J Cell Biol 2002; 156:17-21.
11. Lagos-Quintana M, Rauhut R, Lendeckel W, Tuschl T. Identification of novel genes coding for small expressed RNAs. Science 2001; 294:853-8.
12. Lau N C, Lim L P, Weinstein E G, Bartel D P. An abundant class of tiny RNAs with probable regulatory roles in Caenorhabditis elegans. Science 2001; 294:858-62.
13. Lee R C, Ambros V. An extensive class of small RNAs in Caenorhabditis elegans. Science 2001; 294:862-4.
14. Borchert G M, Lanier W, Davidson B L. RNA polymerase III transcribes human microRNAs. Nat Struct Mol Biol 2006; 13:1097-101.
15. Zeng Y. Principles of micro-RNA production and maturation. Oncogene 2006; 25:6156-62.
16. Lee Y, Ahn C, Han J, Choi H, Kim J, Yim J, et al. The nuclear RNase III Drosha initiates microRNA processing. Nature 2003; 425:415-9.
17. Bernstein E, Caudy A A, Hammond S M, Hannon G J. Role for a bidentate ribonuclease in the initiation step of RNA interference. Nature 2001; 409:363-6.
18. Kim V N. MicroRNA precursors in motion: exportin-5 mediates their nuclear export. Trends Cell Biol 2004; 14:156-9.
19. Hammond S M, Bernstein E, Beach D, Hannon G J. An RNA-directed nuclease mediates post-transcriptional gene silencing in Drosophila cells. Nature 2000; 404:293-6.
20. Bentwich I, Avniel A, Karov Y, Aharonov R, Gilad S, Barad O, et al. Identification of hundreds of conserved and nonconserved human microRNAs. Nat Genet 2005; 37:766-70.
21. Berezikov E, Guryev V, van de Belt J, Wienholds E, Plasterk R H, Cuppen E. Phylogenetic shadowing and computational identification of human microRNA genes. Cell 2005; 120:21-4.
22. Lu J, Getz G, Miska E A, Alvarez-Saavedra E, Lamb J, Peck D, et al. MicroRNA expression profiles classify human cancers. Nature 2005; 435:834-8.
23. Stadler B M, Ruohola-Baker H. Small RNAs: keeping stem cells in line. Cell 2008; 132:563-6.
24. Phillips J R, Dalmay T, Bartels D. The role of small RNAs in abiotic stress. FEBS Lett 2007; 581:3592-7.
25. Taganov K D, Boldin M P, Baltimore D. MicroRNAs and immunity: tiny players in a big field. Immunity 2007; 26:133-7.
26. Poy M N, Eliasson L, Krutzfeldt J, Kuwajima S, Ma X, Macdonald PE, et al. A pancreatic islet-specific microRNA regulates insulin secretion. Nature 2004; 432:226-30.
27. Jiang J, Lee E J, Gusev Y, Schmittgen T D. Real-time expression profiling of microRNA precursors in human cancer cell lines. Nucleic Acids Res 2005; 33:5394-403.
28. Park N J, Yu T, Nabili V, Brinkman B M, Henry S, Wang J, Wong D T. RNAprotect saliva: An optimal room-temperature stabilization reagent for the salivary transcriptome. Clin Chem 2006; 52:2303-4.
29. El-Hefnawy T, Raja S, Kelly L, Bigbee W L, Kirkwood J M, Luketich J D, Godfrey T E. Characterization of amplifiable, circulating RNA in plasma and its potential as a tool for cancer diagnostics. Clin Chem 2004; 50:564-73.
30. Tsui N B, Ng E K, Lo Y M. Stability of endogenous and added RNA in blood specimens, serum, and plasma. Clin Chem 2002; 48:1647-53.
31. Del Prete M J, Robles M S, Guao A, Martinez A C, Izquierdo M, Garcia-Sanz J A. Degradation of cellular MRNA is a general early apoptosis-induced event. FASEB J 2002; 16:2003-5.
32. Scott G K, Goga A, Bhaumik D, Berger C E, Sullivan C S, Benz C C. Coordinate suppression of ERBB2 and ERBB3 by enforced expression of micro-RNA miR-125a or miR-125b. J Biol Chem 2007; 282:1479-86.
33. Yang H, Kong W, He L, Zhao J J, O'Donnell J D, Wang J, et al. MicroRNA expression profiling in human ovarian cancer: miR-214 induces cell survival and cisplatin resistance by targeting PTEN. Cancer Res 2008; 68:425-33.
34. Iorio M V, Visone R, Di Leva G, Donati V, Petrocca F, Casalini P, et al. MicroRNA signatures in human ovarian cancer. Cancer Res 2007; 67:8699-707.
35. Tran N, McLean T, Zhang X, Zhao C J, Thomson J M, O'Brien C, Rose B. MicroRNA expression profiles in head and neck cancer cell lines. Biochem Biophys Res Commun 2007; 358:12-7.

What is claimed is:

1. A method for identifying a micro RNA (miRNA) marker for a human disease state of interest, comprising:
obtaining a human saliva sample from a subject having the disease state and inhibiting the RNAses in the sample;
amplifying said miRNA to provide nucleic acid amplification products of said miRNA and detecting said amplification products;
comparing the amount of miRNA amplification products detected in the sample from the subject having the disease to miRNA amplification products detected for a control sample which came from a subject not having the disease state; thereby identifying whether the miRNA in the saliva sample is differentially expressed between the subject having the disease and the control subject.

2. The method of claim 1, wherein said disease state is selected from cancers, autoimmune diseases, metabolic disorders, diabetes and neurological disorders.

3. The method of claim 1, wherein the RNAse in the sample is inhibited by contacting the sample with an RNAlater™ composition.

4. The method of claim 1, wherein the comparing compares the relative amount of miRNA amplification products detected in a plurality of samples from a corresponding plurality of subjects having the disease state to miRNA amplification products detected for a plurality of control samples which come from a corresponding plurality of subjects not having the disease state; thereby identifying whether the miRNA in the saliva sample is differentially expressed between the subjects having the disease and the control subjects.

5. The method of claim 1, wherein the miRNA marker is selected from the group consisting of:

| | |
|---|---|
| hsa-mir-16 | Let-7b |
| hsa-mir-19b | hsa-mir-26a |
| hsa-mir-24 | hsa-mir-30c |
| hsa-mir-26b | hsa-mir-30a-3p |
| hsa-mir-30e-3p | hsa-mir-30e-5p |
| hsa-mir-92 | hsa-mir-125a |
| hsa-mir-146a | hsa-mir-140 |
| hsa-mir-146b | hsa-mir-155 |
| hsa-mir-150 | hsa-mir-181 |
| hsa-mir-191 | hsa-mir-195 |
| hsa-mir-200c | hsa-mir-197 |
| hsa-mir-203 | hsa-mir-222 |
| hsa-mir-223 | hsa-mir-320 |
| hsa-mir-200a | hsa-mir-342 |
| hsa-mir-142-3p | hsa-mir-375 |
| hsa-mir-93. | |

6. The method of claim 1, wherein the miRNA is miR-200a, miR-125a, miR-142-3p, or miR-93.

7. The method of claim 1, wherein the human saliva sample is a cell-free fluid phase portion of saliva.

8. The method of claim 1, wherein the disease state is a disease state of the head or neck, oral pharyngeal cavity.

9. The method of claim 1, wherein the disease state is a disease state of the tongue.

10. A method for diagnosing or providing a prognosis for a disease state in a subject, said method comprising (a) detecting in a saliva sample from the subject the level of a micro RNA (miRNA) associated with the disease state, and determining whether the level is increased or decreased when compared to a standard control, thereby providing the diagnosis or the prognosis.

11. The method of claim 10, wherein the disease state is oral cancer.

12. The method of claim 10, wherein the diagnosis is provided.

13. The method of claim 10, wherein the prognosis is provided.

14. The method of claim 10, wherein step (a) comprises an amplification reaction.

15. The method of claim 14, wherein the amplification reaction is a polymerase chain reaction (PCR).

16. The method of claim 10, wherein the saliva sample is whole saliva.

17. The method of claim 10, wherein the saliva sample is saliva supernatant.

18. The method of claim 10, wherein the miRNA is miR-200a, miR-125a, miR-142-3p, or miR-93.

19. The method of claim 18, wherein the miRNA is miR-200a or miR-125a and the miRNA level is decreased from the standard control.

20. The method of claim 10, wherein the oral cancer is oral squamous cell carcinomas (OSCC).
